(19) United States
(12) Patent Application Publication
(10) Pub. No.: US 2009/0246230 A1
(43) Pub. Date: Oct. 1, 2009

(54) Compositions and methods for inducing tumor resistance

(75) Inventors: Thomas Wagner, Greenville, SC (US); Yanzhang Wei, Greer, SC (US)

Correspondence Address: FOLEY AND LARDNER LLP;SUITE 500, 3000 K STREET NW, WASHINGTON, DC, 20007, US

(73) Assignee: Greenville Hospital System

(21) Appl. No.: 12/320,214

(22) Filed: Jan. 21, 2009

Related U.S. Application Data
(60) Provisional application No. 61/006,579, filed on Jan. 22, 2008.
(60) Provisional application No. 61/035,874, filed on Mar. 12, 2008.

(51) Int. Cl. A61K 39/00

(52) U.S. Cl. 424/277.1

(57) ABSTRACT

The invention provides a combined whole cell tumor vaccine for inducing tumor resistance in patients and treating cancer.

### CROSS-REFERENCE TO RELATED PATENT APPLICATIONS

This application claims priority from U.S. Provisional Application No. 61/006,579, filed Jan. 22, 2008, incorporated herein by reference in its entirety. In addition, this application claims priority from U.S. Provisional Application No. 61/035,874, filed Mar. 12, 2008, also incorporated herein by reference in its entirety.

### FIELD OF THE INVENTION

Cancer is characterized by uncontrolled growth and spread of abnormal cells. Because tumor cells are derived from normal cells, the host immune system does not recognize tumor cell antigens as foreign. Further, some tumor cells have developed ways to escape the host immune defense system, by eliminating antigens or reducing the number of receptors on the surface of the cell. The present invention is directed to methods and composition strategies for inducing tumor resistance.

### BACKGROUND OF THE INVENTION

Melanoma is a malignant skin cancer that originates in melanocytes. If detected and treated early, it is nearly 100 percent curable. Without early treatment the cancer can advance, spread and be fatal. Melanoma is the skin cancer that causes the most deaths. Superficial spreading melanoma is the most common type of melanoma, especially among young people. This melanoma affects the top layer of the skin for a fairly long time before penetrating more deeply. Lentigo maligna is found most often in the elderly who are chronically exposed to the sun. Acral lentiginous melanoma also spreads superficially before penetrating more deeply and is the most common melanoma in African-Americans and Asians, and the least common among Caucasians. Nodular melanoma is usually invasive at the time it is first diagnosed and is the most aggressive form of melanoma.

Staging of melanoma is based on the thickness of the tumor, known as Breslow's thickness, and the presence of microscopic ulceration, indicating that the epidermis covering the tumor is not intact. Breslow's thickness measures the distance between the upper layer of the epidermis and the deepest point of the tumor's penetration. Very thin tumors (less than 1.0 millimeter) are classified according to Clark's level of invasion, based on the number of layers of skin penetrated by the tumor.

Spreading of melanoma to a lymph node closest to the primary tumor, i.e., the sentinel node, is determined by lymphoscintigraphy, a procedure based on injecting a small amount of radioactive substance to trace a blue dye through the lymphatic fluid, followed by biopsy.

Generally speaking, in stage Ia the tumor is less than 1.0 mm in Breslow's thickness without ulceration and is in Clark's level II or III. In stage Ib the tumor is less than 1.0 mm in Breslow's thickness with ulceration and/or Clark's level III or IV, or it is 1.01-2.0 mm in thickness without ulceration, and may have spread to the closest lymph nodes. In stage Ia the tumor is 1.01-2.0 mm in Breslow's thickness with ulceration, or is 2.01-4.0 mm in thickness without ulceration. In stage IIb the tumor is 2.01-4.0 mm in Breslow's thickness with ulceration, or is greater than 4.0 mm in thickness without ulceration. In stage IIc the tumor is greater than 4.0 mm in Breslow's thickness with ulceration. In stage III the melanoma has spread to the local or regional lymph nodes. Transit or satellite metastases may form in the skin and subcutaneous tissues. In stage IV the melanoma has metastasized to distant lymph nodes or to internal organs, most often the lungs, the liver, the brain, the bone and the gastrointestinal tract.

Surgical excision to remove the melanoma is the treatment of choice. For patients with stages III and IV disease, surgery may be followed by adjuvant chemotherapy. Clinical trials of various melanoma vaccines are underway with patients whose disease is in stages III and IV. Immunotherapy with interferon-alpha has been shown to improve five-year survival of stage III patients, however, significant side effects limit its use.

Another significant health concern is lung cancer, which is one of the most common cancers. It accounts for approximately 15% of all cancer diagnoses and 29% of all cancer deaths. It is the second most diagnosed cancer in men and women after prostate and breast cancer, respectively, but it is the number one cause of death from cancer each year in both men and women. Because lung cancer can take years to develop, it is mostly found in older people. The average age of a person diagnosed with lung cancer is 69 years.

The two main types of lung cancer are non-small cell lung cancer (NSCLC) and small cell lung cancer (SCLC), a more aggressive type of cancer. About 75-80% of all people diagnosed with lung cancer have non-small cell lung cancer. Non-small cell lung cancer includes adenocarcinoma, squamous carcinoma (formerly known as epidermoid carcinoma) and large cell carcinoma. Diagnosis and staging of lung cancer determine the course of treatment. Diagnostic tests include a chest x-ray, computerized tomography (CT), magnetic resonance imaging (MRI), positron emission tomography (PET) scanning, sputum cytology and a biopsy.

Staging classifies the characteristics of the tumor and the degree of spreading. For non-small cell lung cancer, stage 0, also known as carcinoma in situ, is lung cancer found only in the layer of cells lining the air passages. If the location of the tumor can be detected, minimally invasive surgery may be used at this stage to locally remove the tumor and preserve lung function. In stage IA, the cancer is smaller than 3 centimeters, has not spread to the membranes that surround the lungs, the lymph nodes or any other distant organ. In stage IB, the tumor is larger than 3 centimeters but has not spread to the lymph nodes or a distant site. In stage IIA, the tumor is no larger than 3 centimeters, has not spread to the membranes that surround the lungs, and does not affect the main branches of the bronchi. However, the tumor has spread to the lymph nodes within the cancerous lung but not to any distant sites. In stage IIB, the cancer is larger than 3 centimeters and has spread to the membranes that surround the lung and/or the lymph nodes within the same cancerous lung, the chest wall, the diaphragm, the membranes that surround the space between the lungs, or the membranes that surround the sac of the heart without involvement of lymph nodes or distant organs. In stage IIIA, the cancer is confined to the lung itself and the lymph nodes around the windpipe or in the mediastinum on the same side as the cancerous lung. In stage IIIB, the tumor is of any size and has spread to the bronchus, trachea, esophagus, backbone, or the fluid in the space surrounding the lung, and the lymph nodes near the collarbone on either side and/or the lymph nodes within the lung or mediastinal lymph nodes on the side that is opposite the cancerous lung. In stage IV, the cancer has spread from the lungs to other organs, such as the liver, brain, or bone.

Therapeutic cancer vaccines, including antigen vaccines and whole tumor cell vaccines, are designed to treat cancer by stimulating the immune system to recognize and attack cancer cells without damaging normal cells (Pardoll, DM. Nat. Med. 4 (5 Suppl.): 525-531, 1998; Jager E., et al. Curr. Opin. Immunol. 14: 178-182, 2002; Schmitz M., et al. Trends Immunol. 23: 428-429, 2002; Lysaght J. and Todryk S., Curr. Opin. Invest. Drugs 4: 716-721, 2003). Antigen vaccines trigger an immune response by using only unique antigens found on cancer cells (Van Der Bruggen P., et al. Immunol. Rev. 188: 51-64, 2002.). These antigens may be specific to an individual, a type of cancer, or several types of cancer. Whole tumor cell vaccines present the advantage of immunizing the patient with diverse tumor antigens, without depending on the identification of a specific antigen (Lysaght J. and Todryk S. Curr. Opin. Invest. Drugs 4: 716-721, 2003; Ward S., et al. Cancer Immunol. Immunother. 51: 351-357, 2002). Whole tumor vaccines may be autologous or allogeneic.

Autologous tumor cell vaccines are derived from cells surgically removed from the patient's own tumor, inactivated to prevent proliferation in vivo, genetically modified when deemed appropriate, and administered to the patient once again. Although these vaccines present the advantage that the tumor cells match the patient's major histocompatibility complex (MHC) molecules and tumor-associated antigens and contain a large number of antigens, autologous vaccines have the disadvantage that they require a separate preparation process for each patient and a long waiting period for the patient because the cells need to be cultured and manipulated before administration. In the alternative, allogeneic tumor cell vaccines are obtained from existing tumor cell lines and are therefore available to a large population of cancer patients. Allogeneic whole cell vaccines contain a range of tumor antigens, some of which are shared by the patient's tumor, and alloantigens, which elicit alloreactive T-helper cell responses (Mitchell, M S. Curr. Opin. Invest. Drugs 3: 140-149, 2002).

Furthermore, strategies to boost the immune system involve combining whole tumor cell vaccines with dendritic cells or an adjuvant, such as Bacille Calmette-Guérin (BCG) (Moton D L., et al. Ann. Surg. 216: 463-482, 1992) and interleukin-2 (IL-2), modifying whole tumor cells with hapten dinitrophenyl (Berd D., et al. J Clin. Oncol. 15: 2359-2370, 1997), or engineering whole tumor cells to secrete hormones that stimulate the immune response, such as granulocyte macrophage colony stimulating factor (GM-CSF) (Simons J W., et al. Cancer Res. 59: 5160-5168, 1999; Hege K M., et al. Inter. Rev. Immunol. 25: 321-352, 2006), or to express B7 co-stimulatory molecules (Parmiani G., et al. Hum. Gene Ther 11: 1269-1275, 2000; Galili, U. Cancer Immunol. Immunother. 53: 935-945, 2004).

Although significant advances through molecular biology in the identification of tumor antigens and their production in recombinant and synthetic form have allowed many sophisticated approaches in cancer treatment, the immunogenic success of tumor cell vaccines ultimately depends on major histocompatibility complex (MHC) expression on antigen-presenting cells and the recognition of tumor antigens as “foreign” by the host immune system. And because tumor cells have developed several ways to escape the host immune response, no single treatment regimen is perfect. Thus, there is always a need in the art for improved treatment options for cancer patients, including melanoma and lung cancer patients. The present invention satisfies that need.

The present inventors unexpectedly discovered that vaccination with inactivated primary tumor cells and inactivated S180 tumor cells is effective in treating cancer, including melanoma and lung cancer, and in inducing tumor resistance. In particular, the inventors of this application have surprisingly discovered that co-administration or cell fusions of an inactivated S-180 cell with a primary tumor cell greatly enhances the potency of the tumor vaccine.

Although Cui (Cui, Z., et al. Proc. Natl. Acad. Sci. U.S.A., 100: 6682-6687, 2003) and Hicks (Hicks, A M., et al. Proc. Natl. Acad. Sci. U.S.A., 103: 7753-7758, 2006) describe a germ line-transmissible spontaneous regression (SR)/complete resistance (CR) trait in BALB/c mice which confers in vitro resistance to S180 cells and other cancer cells, these SR/CR mice were obtained from a BALB/c founder mouse that was initially identified because of its failure to develop ascites upon injection of S180 cells. In fact, Cui reports that the SR/CR trait is likely to be linked to one of the 19 mouse autosomes, but not to the X or Y chromosome, and Hicks demonstrates that the SR/CR trait is completely transferable to wild type (WT) recipient mice. This is in contrast to the teachings in the present invention, which disclose compositions and methods for inducing tumor resistance in vivo, regardless of genetic make-up in the patient being treated.

### SUMMARY OF THE INVENTION

To this end, the invention provides methods for inducing tumor resistance in a patient having a tumor comprising administering to the patient a vaccine comprising inactivated whole tumor cells and inactivated S180 cells or fragments thereof, including cell membranes isolated from inactivated S180 cells and S180 tumor cell lysates. The patient to be treated may suffer from a number of cancers selected from the group consisting of melanoma, sarcoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer. In a preferred embodiment, the patient suffers from melanoma or lung carcinoma. In one aspect of the invention, the whole tumor cells are autologous. In another aspect of the invention, the whole tumor cells are allogeneic. In one embodiment the vaccine comprises a mixture of inactivated whole tumor cells and inactivated S180 cells. In a different embodiment the vaccine comprises cell fusions of inactivated whole tumor cells fused with inactivated S180 cells.

In a further embodiment, the invention provides a method for treating cancer in a patient comprising administering to the patient a vaccine that comprises inactivated whole tumor cells and inactivated S180 cells or fragments thereof, including cell membranes isolated from inactivated S180 cells. The patient to be treated may suffer from a number of cancers selected from the group consisting of melanoma, sarcoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer. In a preferred embodiment, the patient suffers from melanoma or lung carcinoma. In one aspect of the invention, the whole tumor cells are autologous. In another aspect of the invention, the whole tumor cells are allogeneic. In one embodiment the vaccine comprises a mixture of inactivated whole tumor cells and inactivated S180 cells. In a different embodiment the vaccine comprises cell fusions of inactivated whole tumor cells fused with inactivated S180 cells.

In yet another embodiment, the invention provides methods of inducing an immune response in a patient comprising administering to the patient a vaccine that comprises inactivated whole tumor cells and inactivated S180 cells or fragments thereof, including cell membranes isolated from inactivated S180 cells. The patient to be treated may suffer from a number of cancers selected from the group consisting of melanoma, sarcoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer. In a preferred embodiment, the patient suffers from melanoma or lung carcinoma. In one aspect of the invention, the whole tumor cells are autologous. In another aspect of the invention, the whole tumor cells are allogeneic. In one embodiment the vaccine comprises a mixture of inactivated whole tumor cells and inactivated S180 cells. In a different embodiment the vaccine comprises cell fusions of inactivated whole tumor cells fused with inactivated S180 cells.

In an additional embodiment, the present invention provides a therapeutic vaccine that comprises inactivated whole tumor cells and inactivated S180 cells or fragments thereof, including cell membranes isolated from inactivated S180 cells. The tumor cells may be melanoma cells, sarcoma cells, lung carcinoma cells, breast cancer cells, bladder cancer cells, colon cancer cells, rectal cancer cells, endometrial cancer cells, kidney cancer cells, pancreatic cancer cells, prostate cancer cells, skin cancer (nonmelanoma) cells and thyroid cancer cells. In a preferred embodiment, the tumor cells are melanoma cells or lung carcinoma cells. In one aspect of the invention, the whole tumor cells are autologous. In another aspect of the invention, the whole tumor cells are allogeneic. In one embodiment the vaccine comprises a mixture of inactivated whole tumor cells and inactivated S180 cells. In a different embodiment the vaccine comprises cell fusions of inactivated whole tumor cells fused with inactivated S180 cells.

In a preferred embodiment, the whole tumor cells and the S180 cells or fragments thereof are inactivated by irradiation. In a specific embodiment, the whole tumor cells and the S180 cells or fragments thereof are irradiated with radiation of about 25 to about 100 Gy. In another preferred embodiment, the whole tumor cells and the S180 cells or fragments thereof are inactivated with mitomycin. Preferably, the amount of mitomycin is about 40 μg/ml. See, for example, Kruisbeek, A M., et al. CURRENT PROTOCOLS IN IMMUNOLOGY Unit 3.12 (8) (1991) (eds J. Coligan, A. Kruisbeek, D. Margulies, E. Shevach & W. Strober), John Wiley & Sons, New York, N.Y., for cell inactivation methods.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE DRAWINGS

FIG. 1 shows that vaccination with irradiated S180 cells rejects live S180 tumor cells in syngeneic and allogeneic mice. Syngeneic Swiss Webster (CFW) mice (A), allogeneic C57BL/6J mice (B), or allogeneic BALB/c mice (C) were intra-peritoneally (i.p.) injected with inactivated S180 cells (1×10⁶ cells/mouse/day) on days 1, 2, 3, 10, and 17, and on day 24 all the mice were i.p. injected with 5×10⁵ live S180 cells. The survival of these mice were monitored and graphed using the GraphPad Prism 4 software.

FIG. 2 shows the effects of vaccination with inactivated S180 cells in different mouse models. Nude mice (A), B cell knockout mice (B), C57BL/6J mice (C) and beige mice (D) were i.p. injected with inactivated S180 cells (1×10⁶ cells/mouse/day) on days 1, 2, 3, 10, and 17, and on day 24 all the mice were i.p. injected with 5×10⁵ live S180 cells. On day 23, one day before live tumor injection, the vaccinated C57BL/6J mice were i.p. injected with either rat anti-mouse CD3 antibody (100 μg/mouse) or rat IgG (100 μg/mouse). The survival of these mice was monitored after live tumor injection and graphed using the GraphPad Prism 4 software.

FIG. 3 shows the effects of inactivated S180 cells as adjuvants. C57BL/6J mice were i.p. injected with (A) 0.2 ml PBS, (B) inactivated S180 cells (1×10⁶ cells/mouse/day), (C) inactivated LL2 cells (1×10⁶ cells/mouse/day), (D) a mixture of inactivated S180 cells and inactivated LL2 cells in a ratio of 1:1 (2×10⁶ cells/mouse/day), or (E) inactivated hybrids from the fusion of S180 cells and LL2 cells (2×10⁵ cells/mouse/day) on days 1, 2, 3, 10, and 17, and on day 24 all the mice were i.p. injected with 5×10⁵ live S180 cells. The survival of these mice was monitored after live tumor injection and graphed using the GraphPad Prism 4 software.

FIG. 4 shows the level of TC-1 cell-specific IgGs in sera from mice vaccinated with PBS (4A), inactivated S180 cells (4B), inactivated TC-1 cells (4C), or combined inactivated S180-TC-1 cells. The sera were collected one week after the completion of the 3/1/1 vaccine protocol and pooled for each group (4 mice/group). The sera were diluted 1:10 with PBS and 100 μl of diluted sera from each group were used to stain two hundred thousand TC-1 tumor cells for 30 min on ice. The cells were further stained with goat anti-mouse IgG-PE and analyzed on FACS Calibur.

FIG. 5 shows examples of lungs dissected from C57BL/6J mice which were first administered 2×10⁵ live TC-1 tumor cells by i.v. injection and three days later were treated with PBS (5A), inactivated S180 cells (5B), inactivated TC-1 cells (5C) or the combined inactivated S180-TC-1 cells (5D) on days 3, 4, 5, 10, 15 and 20.

### DETAILED DESCRIPTION OF THE INVENTION

### Definitions

As used herein, the term “isolated” means that the referenced material is removed from the natural environment in which it is normally found. In particular, isolated biological material is free of cellular components. An isolated organelle, cell, or tissue is removed from the anatomical site in which it is found in an organism. An isolated material may be, but need not be, purified.

A “patient” as referenced herein is a human or a non-human animal who may receive the vaccines of the present invention. Preferably the patient is a human. The invention, however, is also contemplated for veterinary medicine, particularly for treatment of domestic pets (dogs, cats), and livestock (horses, cows, pigs, etc.).

An “anti-tumor response” as used herein is at least one of the following: tumor necrosis, tumor resistance, tumor regression, tumor inflammation, tumor infiltration by activated T lymphocytes, increased IgG level in serum, elevated cytotoxic T lymphocyte (CTL) activity, a delayed-type hypersensitivity (DTH) response and a clinical response.

### Vaccine Compositions

One embodiment of the present invention is a vaccine comprising an inactivated tumor cell and an inactivated S180 cell. The tumor cell is a tumor cell preferably isolated from the patient to be treated, and can be a tumor cell from a melanoma, lung cancer, or other solid tumor. For example, solid tumor cells that can be used in the preparation of the vaccines described herein include sarcomas, carcinomas, and other tumors such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and hematologic cancers. The S180 cell is isolated from a mouse sarcoma tumor cell line and can be a whole S180 cell, a fragment of S180 cell, or S180 cell protein extract/cell lysate. For protein extracts, S180 cells are digested with trypsin, washed twice with PBS and resuspended in serum-free medium at a density of 5×10⁶/ml. The cells are then irradiated at 10,000 rads and subjected to freezing at −80° C. and thawing for four cycles. The resulting lysates are spinned for 10 minutes at 300 g and the supernatant is collected and filtered through a 0.2 μm. Protein concentration is determined by spectrophotometric analysis.

The tumor cell and the S180 cell of the present invention are “inactivated” so as to destroy or damage a cell's tumor producing capacity. This usually involves destroying the cell's ability to proliferate or divide. Conventional methods of rendering cells incapable of cell growth and division are known to a person of skill in the art. For example, cells may be irradiated prior to use. Alternatively, the cells may be inactivated with mitomycin. See, for example, Kruisbeek, A M., et al. CURRENT PROTOCOLS IN IMMUNOLOGY Unit 3.12 (8) (1991) (eds J. Coligan, A. Kruisbeek, D. Margulies, E. Shevach & W. Strober), John Wiley & Sons, New York, N.Y., for cell inactivation methods.

The vaccine of the present invention can comprise an inactivated tumor cell and an inactivated S180 cell as individual and distinct cells, or as a cell fusion. In other words, the vaccine described herein can comprise two separate cell types, i.e., an irradiated S180 cell and an irradiated tumor cell from a patient, or a hybrid cell, which is a fusion of an irradiated S180 cell and a tumor cell from a patient. Inactivated whole tumor cells or tumor cell extracts are suitable for use in the present invention.

### Methods of Making

The vaccines of the present invention can be made according to known methods. For example, the compositions of the present invention are prepared from S180 cells and tumor cells surgically resected from a cancer patient in the course of a cancer treatment regimen. The tumor cells should preferably originate from the same type of cancer as that to be treated, and are even more preferably syngeneic (e.g., autologous or tissue-type matched). For purposes of the present invention, syngeneic refers to tumor cells that are closely enough related genetically that the immune system of the intended recipient will recognize the cells as “self,” e.g., the cells express the same or almost the same complement of MHC molecules. Another term for this is “tissue-type matched.” For example, genetic identity may be determined with respect to antigens or immunological reactions, and any other methods known in the art. A syngeneic tumor cell can be created by genetically engineering a tumor cell to express the required MHC molecules.

Although allogeneic tumor cells are also suitable for the present invention, most preferably the tumor cells originated from the same patient who is to be treated. The tumor cells may be, but are not limited to, autologous cells dissociated from biopsy or surgical resection specimens, or from tissue culture of such cells.

The tumor cells to be used in the present invention are preferably prepared as follows. Briefly, the cells are extracted by dissociation, such as by enzymatic dissociation with collagenase and DNase, by mechanical dissociation in a blender, by teasing with tweezers, using mortar and pestle, cutting into small pieces using a scalpel blade, and the like. After the tumor cells are isolated from the tumor tissue, they are characterized by FACS or ICC and/or purified if necessary, irradiated, and then cryopreserved.

Cell fusions between irradiated S180 cells and irradiated tumor cells can be prepared according to known methods. See, for example, Kruisbeek, A M., CURRENT PROTOCOLS IN IMMUNOLOGY Unit 3.14.2 (1997) (eds J. Coligan, A. Kruisbeek, D. Margulies, E. Shevach & W. Strober), John Wiley & Sons, New York, N.Y., for cell fusion methods. As an example, the two cells are brought together under conditions that promote cell fusion, and the resulting hybrid cell is purified without metabolic selection. Such fusion-promoting conditions are well known to the artisan, and typically involve the addition of an agent that promotes cell fusion. These agents are thought to work by a molecular crowding mechanism to concentrate cells to an extent that they are in close enough proximity to cause fusion of cell membranes. While the invention contemplates any agent that meets these characteristics, exemplary useful agents are polymeric compounds, like polyethylene glycols. An effective amount of such an agent generally will be from about 20% to about 80% (w/v). A preferred range is from about 40% to about 60%, with about 50% being more preferred.

Another suitable method for fusing the tumor cells and S180 cells is by electrofusion, a technique known in the art. Electroporation is the use of electrical fields to induce a reversible breakdown of the cell's lipid bilayer membrane, causing temporary pore formation through which various molecules such as proteins, peptides, DNA or RNA may enter the cell. Electrofusion is the result of an intensive electroporation with membrane breakdown of juxtaposed cells in an inhomogeneous electrical field and consecutive fusion by mutual resealing, Teissie et al., Biophys J 74: 1889-1898 (1998). Because the size of the membrane pores is directly related to the strength of the electric field, electrofusion settings are usually characterized by higher field strengths (1000-1875V/cm) than electroporation settings (250-1025V/cm), Shimizu et al., J Immunother 27: 265-72 (2004); Kjaergaard et al., Cell Immunol 225: 65-74 (2003); Orentas et al., Cell Immunol 213: 4-12 (2001); Meldrum et al., Biochem Biophys Res Comm 256: 235-239 (1999).

If dyes, such as fluorescent dyes, are used to label the cells prior to the fusion process, the fused cells can be separated by fluorescence activated cell sorting (FACS), and the like. Dyes useful according to the invention have the characteristic of associating with a cell for a time sufficient to detect them in such association. In addition, useful dyes do not substantially diminish cell viability, with greater than about 50% cell viability being preferred. Typically, they are fluorescent dyes. One useful class of dyes comprises the so-called “cyanine” dyes. Cyanine dyes come in a variety of types that fluoresce at different wavelengths such that they can be individually or jointly detected when associated with a cell. Some exemplary cyanine dyes are found in Horan et al., U.S. Pat. Nos. 4,783,401 (1998), 4,762,701 (1988) and 4,859,584 (1989), the structures of which are hereby specifically incorporated by reference.

Two particularly useful cyanine dyes are PKH26-GL and PKH67-GL, which may be obtained from the Sigma Chemical Co. These dyes are preferred because they have been widely studied and used. For instance, they have been used in animal studies in vivo for cell trafficking studies. Horan et al., Nature 1989; 340, 167-168; Horan et al., Methods Cell Biol. 1990; 33, 469-490; Michelson et al., Proc. Natl. Acad. Sci. USA 1996; 93, 11877-11882. In laboratory animals, these dyes have been shown not to affect cell growth or function and not to migrate from the cells stained with these dyes to other cells (Horan et al., 1989), thus have low toxicity, a desirable quality for in vivo applications.

Dyes employed in vivo in accordance with the present invention should be free of endotoxin, as measured, for example, by the Limulus amaebocyte (LAL) assay. Typically, when the measured endotoxin level is less than about 1 ng/μg dye, and preferably less than about 0.1 ng/μg dye, then the dye is considered “endotoxin-free.”

More generally, the dyes are essentially pyrogen-free, whether pyrogenicity is contributed by endotoxin or other pyrogens. Thus, a dye is considered “essentially pyrogen free” when the final formulation of hybrid cells labeled with the dye (in a form to be injected into a subject, for example) yields less than about 1 endotoxin unit (EU)/dose, but preferably less than about 0.1 EU/dose and most preferably less than about 0.05 EU/dose. Toxicity thresholds are informed by the fact that most in vivo methods contemplated herein result in less than about 10⁻⁸ g of these dyes, in association with cells, being introduced into a patient when undertaking the inventive methods of treatment.

Conventional cyanine dye labeling methodologies require the presence of cellular stabilizers (osmolarity regulating agents), like sugars (e.g., glucose or mannitol), amino acids and/or certain Goods buffers. See, for example, Horan et al., U.S. Pat. No. 4,783,401 (1998). The inventors discovered that dimethyl sulfoxide (DMSO) can substitute for such stabilizers. In particular, DMSO diluted in a standard culture medium may be used as a solvent for cyanine dyes, and it promotes efficient and stable uptake of dye without substantial loss of cell viability. A generally useful range of DMSO concentration is from about 10 to about 50%, but a preferred range is from about 20 to about 40%. The invention therefore also contemplates methods of labeling cells, and corresponding kits, with cyanine dyes using DMSO in place of the conventional stabilizers.

While the use of dyes for labeling cells is suitable for the present invention, other markers for labeling cells can be used as well and are known in the art. See, for example, Abbas, A K., Lichtman, A H. and Pillai S. CELLULAR MOLECULAR IMMUNOLOGY, Philadelphia, Elsevier, 6th edition, 2007, pp. 399-406.

After formation of the fused cell, it is usually beneficial to isolate it from the un-fused reactant cells. In general, the purification is accomplished in a relatively short period of time, for example, in less than about 24 to 48 hours, after exposure to conditions that promote fusion. In the case of cellular vaccines, for example, this purification substantially increases the potency. As stated above, purification may be accomplished by conventional FACS methodologies, for example.

The vaccines of the present invention may also comprise a pharmaceutically acceptable carrier. The phrase “pharmaceutically acceptable” refers to molecular entities at particular concentrations, and compositions that are physiologically tolerable and do not typically produce an allergic or similar untoward reaction, such as gastric upset, fever, dizziness and the like, when administered to a human or non-human animal. Preferably, as used herein, the term “pharmaceutically acceptable” means approved by a regulatory agency of the Federal or a state government or listed in the U.S. Pharmacopoeia or other generally recognized pharmacopoeia for use in humans or non-human animals.

Additionally, the vaccines described herein may optionally contain conventional vaccine additives like diluents, adjuvants, antioxidants, preservatives and solubilizing agents. The term “adjuvant” refers to a compound or mixture that enhances the immune response to an antigen (Hood et al., IMMUNOLOGY, Second Edition, 1984, Benjamin-Cummings: Menlo Park, Calif., p. 384). While commercially available pharmaceutically acceptable adjuvants are limited, representative examples of adjuvants include Bacille Calmette-Guerin (BCG) the synthetic adjuvant QS-21 comprising a homogeneous saponin purified from the bark of Quillaja saponaria and Corynebacterium parvum (McCune et al., 1979 Cancer 43: 1619). Other adjuvants include Complete and Incomplete Freund's Adjuvant, detoxified endotoxins, mineral oils, surface active substances such as lipolecithin, pluronic polyols, polyanions, peptides, and oil or hydrocarbon emulsions. In some cases, immunostimulatory compounds, as exemplified below, may function as adjuvants.

### Treatment Methods

Described herein is also a method of treating a patient suffering from cancer or inducing tumor resistance in a patient comprising administering to the patient a vaccine comprising inactivated whole tumor cells and inactivated S180 cells. Preferably, the inactivated tumor cells are isolated from the patient to be treated with the vaccine composition of the present invention.

Dosages may be set with regards to weight, and the clinical condition of the patient. The proportional ratio of active ingredient to carrier naturally depend on the chemical nature, solubility, and stability of the compositions, as well as the dosage contemplated. The amount of tumor cells to be formulated in the compositions depend on such factors as the affinity of the inventive composition for cancer cells, the amount of cancer cells present, and the solubility of the composition. The tumor vaccines of the present invention may be administered by any suitable route, including inoculation and injection via, for example, intradermal, intravenous, intraperitoneal, intramuscular, and subcutaneous routes.

Examples of solid tumors that can be treated according to the invention include sarcomas, carcinomas, and other tumors such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In one preferred embodiment, the cancer to be treated is a melanoma, including stage-4 melanoma, lung cancer, ovarian cancer, including advanced ovarian cancer, colon cancer, including colon cancer which has metastasized to the liver, and rectal, colorectal, breast, lung, kidney, and prostate cancers.

Furthermore, the treatment may be augmented by using additional antineoplastic agents in conjunction with the hybrid cells or as part of the administration scheme for the inactivated tumor cells and S180 cells. One class of such agents is immunomodulators. These include cytokines and lymphokines, especially interleukin-2 (IL-2) and IL-2 derivatives, like aldesleukin (Proleukin, Chiron Corp.). The use of IL-2 is preferred because it should further enhance the immune response generated by the compositions described herein. As used herein, “interleukin-2” is used generically to refer to the native molecules and any derivatives or analogs that retain essential interleukin-2 activity, like promoting T cell growth. Other lymphokines and cytokines may also be used as an adjunct to treatment. Examples include interferon gamma (IFN-γ), granulocyte macrophage colony simulating factor (GM-CSF), and the like.

Standard criteria for evaluating treatment response include: complete response, which indicates complete disappearance of all metastases for at least about one month, more preferably for at least about three months, without development of new metastases; partial response, which indicates at least about 30% reduction in the mean diameter of a measurable metastasis for at least about one month, more preferably for at least about three months, without development of new metastases; and a mixed response, which indicates at least about 50% reduction in the mean diameter of a measurable metastasis with concomitant growth of another metastasis. Stable disease indicates more than about 25% increase in the mean diameter of any measurable metastasis. Prolongation of time to relapse or of survival are both also indicative of an effective method of treatment.

The invention is further described by reference to the following examples, which are provided for illustration only. The invention is not limited to the examples but rather includes all variations that are evident from the teachings provided herein.

### EXAMPLES

### Example 1

### Mice and Cell Lines

Female C57BL/6J mice, female BALB/c mice, female B cell knockout mice ((B6.129S2-Igh-6tmlCgn/J)), female nude mice and beige mice (C57BL6J/LYST<bg-J/J) at 6-8 weeks of age were purchased from Jackson Laboratories (Bar Harbor, Me.). Female CFW mice at 6-8 weeks of age were purchased from Charles River Laboratories (Wilmington, Mass.). The mice were housed in pathogen-free animal facilities. The animal experiments were carried out in accordance with the Guidelines for the Care and Use of Laboratory Animals (NIH Publication Number 85-23) and the institutional guidelines. Four mouse cell lines, B16F0 melanoma cells (ATCC CRL-6322), S180 sarcoma cells (ATCC TIB-66), LL/2 Lewis lung carcinoma cell (ATCC CRL-1642) and TC-1 lung carcinoma cells (ATCC # JHU-1) were used in this study. TC-1 cells were cultured in RPMI 1640 medium with a supplement of 10% FBS, non-essential amino acids, 100 μg/ml gentamicin, and 1 mM sodium pyruvate. The other cell lines were cultured in DMEM medium containing 10% FBS and 100 μg/ml gentamicin at 37° C. with 5% CO₂.

### Example 2

### Vaccination Protocol

For vaccination, mice tumor cell lines (B16F0, LL/2, TC-1 and S180) were inactivated by radiation at 10,000 rads using a Cesium 147 source in the blood center of Greenville Hospital System. Mice were intraperitoneally (i. p.) injected with inactivated tumor cells in 0.2 ml PBS with or without inactivated S180 cells (1×10⁶ cells/mouse, and if with S180 cells, 2×10⁶ cells/mouse in a 1:1 ratio) on days 1, 2, 3, 10 and 17. This vaccination protocol was named as 3/1/1 protocol. On day 24, half million live tumor cells per mouse were either i. p. or intravenously (i. v.) injected. The survival of these mice were monitored and recorded. For intravenous tumor inoculation, pulmonary metastasis was observed 3-4 weeks after tumor cell injection.

TABLE 1
The 3/1/1 Protocol

| | Treatment | |
|---|---|---|
| | 1 × 10⁶ Irradiated B16F0, | Treatment |
| | LL/2 or TC-1 With or | 0.5 × 10⁶ Alive S180 |
| | Without 1 × 10⁶ Irradiated | Cells, Alive B16F0 or |
| Days | S180 cell | Alive LL/2 |
| 1 | X | — |
| 2 | X | — |
| 3 | X | — |
| 10 | X | — |
| 17 | X | — |
| 24 | — | X |

### Example 3

### Inactivated S180 Cell Vaccination of Nude Mice, B. Cell Knockout Mice, Beige Mice and T Cell Depleted Mice

In order to study the roles of different immune cells in the tumor rejection induced by inactivated S180 cell vaccination, nude mice, B cell knockout mice, or beige mice lacking functional natural killer (NK) cells were vaccinated with inactivated S180 cells using the 3/1/1 protocol and one week after the last vaccine injection the mice were challenged with half million of live S180 cells by i.p. injection. In the T cell depletion study, female C57BL/6J mice were vaccinated according to the 3/1/1 protocol, and a day before live tumor injection, the mice were i.p. injected with rat anti-mouse CD3 monoclonal antibody or rat IgG (BD Biosciences, CA) antibody as control (100 μg/mouse). The mice were then challenged with a half million live tumor cells the next day.

### Example 4

### Inactivated S180 Cells as Adjuvants in Tumor Prevention

Three tumor models were used in the preventive study: Lewis lung carcinoma (LL/2), melanoma (B16F0), and lung carcinoma (TC-1). The vaccines were made in two different formats: one million inactivated tumor cells mixed with one million inactivated S180 cells/day/mouse or inactivated hybrids from the fusion of the tumor cells and S180 cells (2×10⁵ cells/day/mouse). The 3/1/1 protocol was employed in this study and half million live tumor cells were used to challenge the mice one week after the last vaccine injection (i.p. for LL/2 cells; i.v. for TC-1 and B16F0 cells). Mice injected with TC-1 or B16F0 were sacrificed between 3 and 4 weeks after tumor injection and tumor nodules on their lungs were counted. Survival was monitored for mice injected with LL/2 cells.

### Example 5

### Vaccination with Inactivated S180 Cells Rejects Live S180 Tumor Cells in Syngeneic and Allogeneic Mice

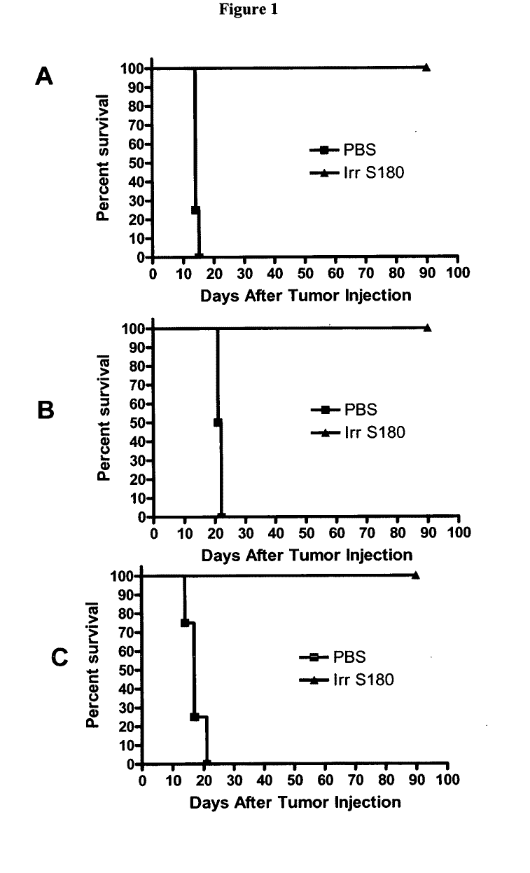

To determine the effect of vaccination with inactivated S180 cells on the response of mice to live S180 cell challenge, eight syngeneic CFW mice, eight allogeneic BALB/c mice and eight allogeneic C57BL/6J mice were vaccinated according to the protocol described above. All the mice vaccinated with S180 cells were tumor free until the end of the study (FIG. 1). In contrast, all mice injected with PBS developed tumors and died within 15 days (CFW mice) or 25 days (C57BL/6J and BALB/c mice). Vaccination with inactivated S180 cells rejects live S180 tumor cell challenge not only in allogeneic C57BL/6J mice (FIG. 1B) or BALB/c mice (FIG. 1C), but also in syngeneic CFW mice (FIG. 1A).

### Example 6

### Serum TC-1 Cell-Specific IgG Staining

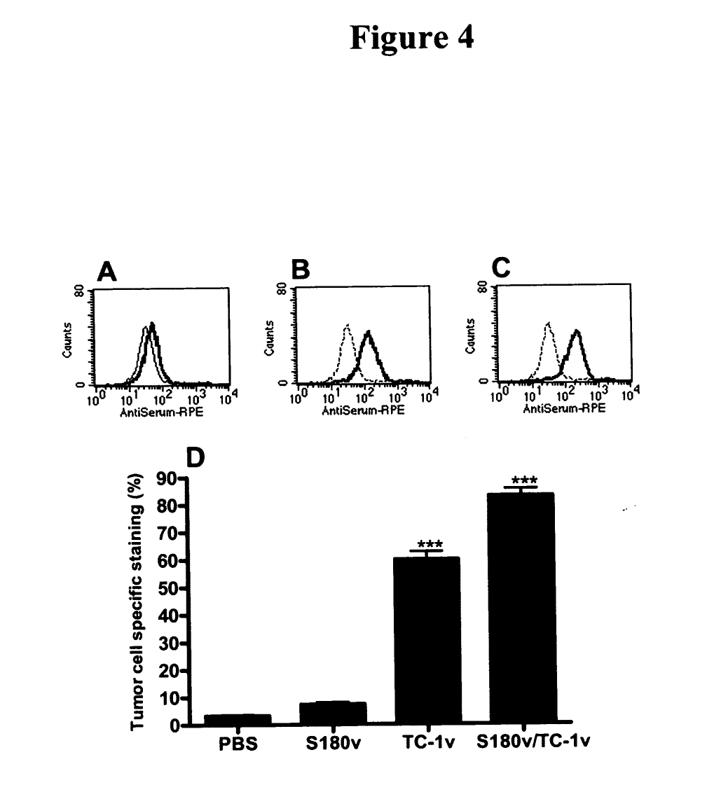

The sera collected from mice that received PBS, S180 vaccine, TC-1 vaccine or the combined S180-TC-1 vaccine were collected one week after the completion of the vaccination protocol and pooled for each group (4 mice/group). The sera were diluted 1:10 with PBS, and 100 μl of the diluted sera from each group were used to stain 2×10⁵ TC-1 cells for 30 minutes on ice. The cells were further stained with goat anti-mouse IgG-PE and analyzed by FACS Calibur. The results are shown in FIG. 4.

S180 vaccine alone did not increase the level of TC-1 cell-specific IgGs compared with sera from PBS injected mice (p>0.05, FIGS. 4A and 4D). Although the TC-1 vaccine increased the level of TC-1 cell-specific IgGs significantly compared to the sera of S180 vaccinated mice (p<0.01, FIGS. 4B and 4D), the combined S180-TC-1 vaccine generated the highest serum level of TC-1 cell-specific IgGs (p<0.01, FIGS. 4C and 4D).

### Example 7

### T and B Cells, but not NK Cells are Required for the Tumor Rejection Induced by Inactivated S180 Cell Vaccination

Several types of cells are involved in the activation of the immune system in mice, including cytotoxic T cell lymphocytes (CTL), B cells and natural killer cells (NK). To determine the type of effector cells in the immune system that are involved in the tumor rejection induced by inactivated S180 cell vaccination, the same vaccination protocol was tested in nude mice, B cell knockout mice, T cell depleted C57BL/6J mice and beige mice. Nude mice lack a thymus and thus cannot generate mature T lymphocytes. Nude mice are therefore unable to form antibodies that require CD4+ helper T cells or activate any cell-mediated immune response that requires CD4+ and/or CD8+ T cells, such as delayed hypersensitivity responses, graft rejections and target killing. B cell knockout (KO) mice are generated by introduction of a non-sense mutation into the transmembrane exon of the IgM heavy chain gene, which results in total deletion of B cells. Similarly, T cell depleted C57BL/6J mice have impaired T lymphocyte function. Beige mice are deficient in NK cells. NK cells are large granular lymphocytes that have activating receptors that activate the NK cells upon binding to a target cell, and killer inhibitory receptors (KIRs) that transmit an inhibitory signal in the presence of class I MHC molecules. Because cells infected by viruses have suppressed class I MHC expression, and cancer cells have reduced or no class I MHC expression, both virus-infected cells and tumor cells are targeted by NK cells. NK cells are also activated by INF-γ and TNF-α.

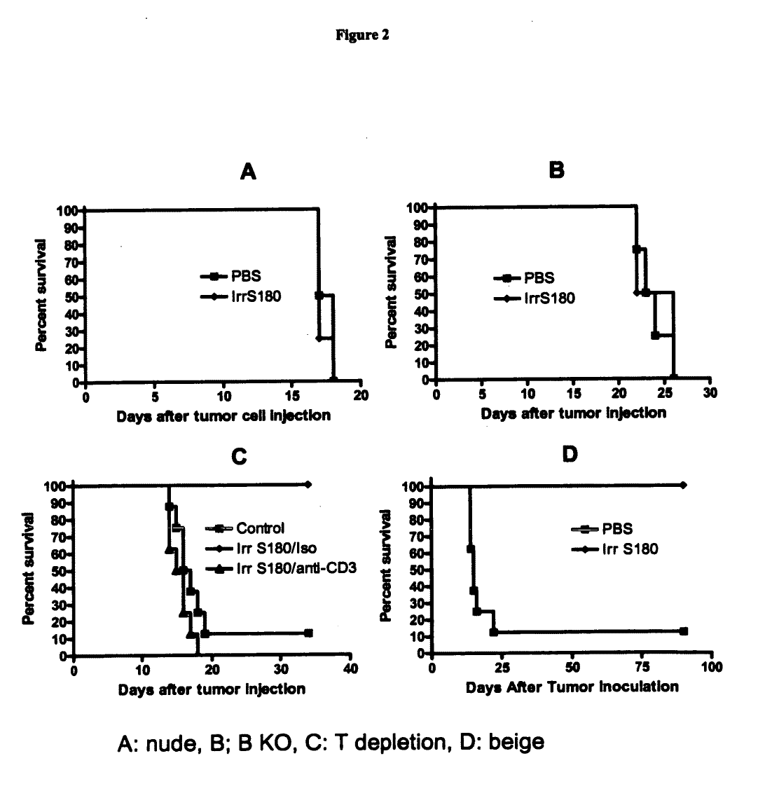

Vaccination of nude mice and B cell KO mice with inactivated S180 cells did not provide the mice with any tumor protection against live S180 cell inoculation. All the nude mice died within 18 days after live S180 tumor injection and there was no difference between the control group and the vaccinated group (FIG. 2A). A similar result was observed in B cell KO mice with the exception that both the control mice and the vaccinated mice lived slightly longer (up to 26 days, FIG. 2B). When their T cells were depleted by anti-CD3 antibody injection, the vaccinated B cell KO mice lost their ability to reject live S180 tumor challenge, developed ascites, and died within 18 days. In contrast, vaccinated mice that received isotype IgG were tumor free (FIG. 2C). Beige mice vaccinated with inactivated S180 cells were able to completely reject live tumor challenge (FIG. 2D). These results clearly demonstrate that CTL and B cells, but not NK cells, are required to provide immunity against cancer.

### Example 8

### Whole Tumor Cell Vaccination with Inactivated S180 Cells as Adjuvants Rejects the Tumor in Various Tumor Models

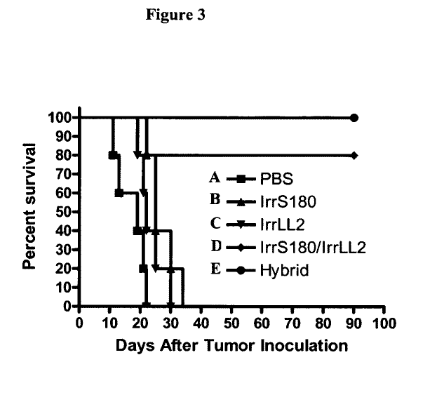

The effect of inactivated S180 cells as adjuvants to whole tumor cell vaccinations was determined in mice affected by Lewis lung carcinoma (LL), melanoma or lung carcinoma. For Lewis lung carcinoma, the tumor cell line LL/2 was used. Five groups of C57BL/6J mice (5 mice each group) were vaccinated according to the 3/1/1 protocol with PBS, inactivated S180 cells, inactivated LL/2 cells, inactivated mixture of S180 cells and LL/2 cells, or inactivated hybrids from S180 and LL/2 cells. One week after the last vaccination, all the mice were i. p. challenged with a half million live LL/2 cells. Mice vaccinated with inactivated LL/2 cells alone lived slightly longer than PBS control mice, but the difference was not statistically significant. Mice vaccinated with inactivated S180 cells alone lived longer than the PBS control group (p<0.01), but all of them died within 34 days. Vaccination of mice with inactivated S180 cells mixed with inactivated LL/2 cells provided 80% of the mice with complete tumor rejection. Vaccination of inactivated hybrids of S180 cells and LL/2 cells provided 100% of the mice with complete tumor rejection, despite the small number of cells used in the vaccine (2×10⁵ cells/mouse) (FIG. 3).

When the same vaccination strategy was tested in two additional tumor models, B16F0 melanoma and TC-1 lung carcinoma cell lines, vaccination of mice with inactivated S180 cells mixed with inactivated B16F0 cells dramatically decreased or completely eliminated lung tumor nodules compared to control mice.

TABLE 2
Pulmonary metastasis of B16F0 tumors in C57BL/6J
mice vaccinated with PBS or inactivated S180 cells
mixed with inactivated whole tumor cells.

| Tumor | Treatment | Pulmonary Metastasis |
|---|---|---|
| B16F0 | PBS | >200, >200, >200, >200, >200 |
| | Mixture of IrrB16F0/IrrS180 | 0, 0, 0, 2, 12 |

Similar results were obtained in the TC-1 tumor model: C57BL/6J mice were vaccinated with PBS, inactivated S180 cells, inactivated TC-1 cells, or the combined vaccine comprising inactivated S180 cells mixed with inactivated TC-1 cells. One week after the last vaccine injection, all the mice were i.v. challenged with 2×10⁵ live TC-1 cells. Four weeks later, the mice were sacrificed and the lung tumor nodules were counted. The results are summarized in Table 3. Although vaccination with allogenic inactivated S180 cells alone did not protect mice against live TC-1 tumor cell challenge compared to the PBS control group (p>0.05), vaccination with syngenic inactivated TC-1 cells alone significantly rejected the live TC-1 tumor challenge when compared to vaccination with inactivated S180 cells (p<0.01), albeit all the mice still developed many tumor nodules ranging from 7 to 88 on their lungs. In contrast, vaccination with combined inactivated S180-TC-1 cells completely protected the mice, with all the eight mice free of any observable tumors. Vaccination of mice with inactivated hybrids of S180 cells and TC-1 cells rendered 100% of the mice tumor free (Table 2).

TABLE 3
Pulmonary metastasis of TC-1 tumors in C57BL/6J mice
vaccinated with PBS, inactivated S180 cells, inactivated
TC-1 cells, inactivated S180 cells mixed with inactivated
whole tumor cells, or inactivated hybrids.

| Tumor | Treatment | Pulmonary Metastasis |
|---|---|---|
| TC-1 | PBS | >200, >200, >200, >200, >200, |
| | | 184, >200, >200 |
| | Inactivated S180 cells | >200, 147, 98, >200, 166, 139, |
| | | >200, 162 |
| | Inactivated TC-1 cells | 88, 31, 56, 49, 72, 7, 49, 22 |
| | Mixture of IrrTC-1/IrrS180 | 0, 0, 0, 0, 0, 0, 0, 0 |
| | Irradiated hybrids | 0, 0, 0, 0, 0, 0, 0, 0 |

These results clearly indicate that inactivated S180 cells induce tumor resistance when used as adjuvants to whole tumor cell vaccines.

### Example 9

### Inactivated S180 Cells as Adjuvants in Tumor Treatment

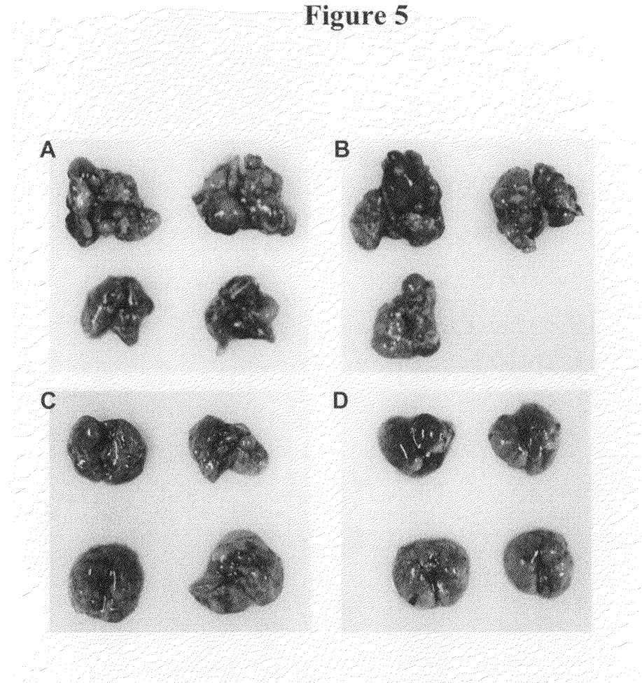

In the treatment study, four groups of C57BL/6J mice (4 mice/group) were i.v. injected with 2×10⁵ live TC-1 tumor cells. Three days later, the tumor-injected mice were treated with PBS, inactivated S180 cells, inactivated TC-1 cells or the combined inactivated S180-TC-1 cells on days 3, 4, 5, 10, 15 and 20. Four weeks after the live tumor cell injection, the mice were sacrificed and the lung tumor nodules counted. The results are summarized in Table 4, and FIG. 5 shows examples of lungs dissected from the mice subjected to the different treatments.

Treatment with inactivated S180 cells did not eradicate existing tumor cells compared to the control group (p>0.05). Although treatment with inactivated TC-1 cells eradicated existing tumor cells to some degree compared to the treatment with inactivated S180 cells (p<0.05), treatment with combined inactivated S180-TC-1 cells significantly improved eradication of existing tumor cells when compared with treatment with inactivated TC-1 cells (p<0.01). Four of the eight mice were tumor free and the other four had only 2, 3, 8, and 11 tumor nodules, respectively.

TABLE 4
Pulmonary metastasis of TC-1 tumors in C57BL/6J mice treated
with PBS, inactivated S180 cells, inactivated TC-1 cells,
or inactivated S180 cells mixed with inactivated whole tumor
cells after live TC-1 tumor cell injection

| Treatment | Pulmonary Metastasis |
|---|---|
| PBS | >200, 67, 136, 89, 113, 147, >200, 88 |
| S180 vaccine* | 103, 73, 125, 57, 134, >200, 84 |
| TC-1 vaccine | 66, 41, 16, 5, 55, 32, 17, 87 |
| Combined vaccine | 0, 0, 8, 0, 2, 11, 0, 3 |

*one mouse died before the counting date.

All of the publications and patent applications and patents cited in this specification are herein incorporated by reference in their entirety.

1. A method for inducing tumor resistance in a patient having a tumor comprising administering to the patient a vaccine comprising inactivated whole tumor cells and inactivated S180 cells, or inactivated fragments thereof.

2. The method of claim 1, wherein the tumor is selected from the group consisting of melanoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer.

3. The method of claim 2, wherein the tumor is melanoma.

4. The method of claim 2, wherein the tumor is lung carcinoma.

5. The method of claim 1, wherein the inactivated whole tumor cells are autologous.

6. The method of claim 1, wherein the inactivated whole tumor cells are allogeneic.

7. The method of claim 1, wherein the whole tumor cells and the S180 cells or fragments thereof are inactivated by irradiation.

8. The method of claim 7, wherein the whole tumor cells and the S180 cells are irradiated with radiation in an amount of about 25 to about 100 Gy.

9. The method of claim 1, wherein the whole tumor cells and the S180 cells are inactivated with mitomycin.

10. The method of claim 9, wherein the amount of mitomycin is about 40 μg/ml.

11. The method of claim 1, wherein the vaccine comprises a mixture of inactivated whole tumor cells and inactivated S180 cells, or fragments thereof.

12. The method of claim 1, wherein the vaccine comprises fusions of inactivated whole tumor cells and inactivated S180 cells, or fragments thereof.

13. A method of treating a patient having cancer comprising administering to the patient a vaccine comprising inactivated whole tumor cells and inactivated S180 cells, or fragments thereof.

14. The method of claim 13, wherein the cancer is selected from the group consisting of melanoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer.

15. The method of claim 14, wherein the cancer is melanoma.

16. The method of claim 14, wherein the cancer is lung carcinoma.

17. The method of claim 13, wherein the inactivated whole tumor cells are autologous.

18. The method of claim 13, wherein the inactivated whole tumor cells are allogeneic.

19. The method of claim 13, wherein the whole tumor cells and the S180 cells or fragments thereof are inactivated by irradiation.

20. The method of claim 19, wherein the whole tumor cells and the S180 cells or fragments thereof are irradiated with radiation in an amount of about 25 to about 100 Gy.

21. The method of claim 13, wherein the whole tumor cells and the S180 cells or fragments thereof are inactivated with mitomycin.

22. The method of claim 21, wherein the amount of mitomycin is about 40 μg/ml.

23. The method of claim 13, wherein the vaccine comprises a mixture of inactivated whole tumor cells and inactivated S180 cells or fragments thereof.

24. The method of claim 13, wherein the vaccine comprises fusions of inactivated whole tumor cells and inactivated S180 cells or fragments thereof.

25. A method of inducing an immune response in a patient comprising administering to the patient a vaccine comprising inactivated whole tumor cells and inactivated S180 cells, or fragments thereof.

26. The method of claim 25, wherein the patient has a tumor selected from the group consisting of melanoma, lung carcinoma, breast cancer, bladder cancer, colon cancer, rectal cancer, endometrial cancer, kidney cancer, pancreatic cancer, prostate cancer, skin cancer (nonmelanoma) and thyroid cancer.

27. The method of claim 26, wherein the tumor is melanoma.

28. The method of claim 26, wherein the tumor is lung carcinoma.

29. The method of claim 25, wherein the inactivated whole tumor cells are autologous.

30. The method of claim 25, wherein the inactivated whole tumor cells are allogeneic.

31. The method of claim 25, wherein the whole tumor cells and the S180 cells or fragments thereof are inactivated by irradiation.

32. The method of claim 31, wherein the whole tumor cells and the S180 cells or fragments thereof are irradiated with radiation in an amount of about 25 to about 100 Gy.

33. The method of claim 25, wherein the whole tumor cells and the S180 cells or fragments thereof are inactivated with mitomycin.

34. The method of claim 33, wherein the amount of mitomycin is about 40 μg/ml.

35. The method of claim 25, wherein the vaccine comprises a mixture of inactivated whole tumor cells and inactivated S180 cells or fragments thereof.

36. The method of claim 25, wherein the vaccine comprises fusions of inactivated whole tumor cells and inactivated S180 cells or fragments thereof.

37. A therapeutic vaccine comprising inactivated whole tumor cells and inactivated S180 cells, or fragments thereof.

38. The therapeutic vaccine of claim 37, wherein the tumor cells are selected from the group consisting of melanoma cells, lung carcinoma cells, breast cancer cells, bladder cancer cells, colon cancer cells, rectal cancer cells, endometrial cancer cells, kidney cancer cells, pancreatic cancer cells, prostate cancer cells, skin cancer (nonmelanoma) cells and thyroid cancer cells.

39. The therapeutic vaccine of claim 38, wherein the tumor cells are melanoma cells.

40. The therapeutic vaccine of claim 38, wherein the tumor cells are lung carcinoma cells.

41. The therapeutic vaccine of claim 37, wherein the inactivated whole tumor cells are autologous.

42. The therapeutic vaccine of claim 37, wherein the inactivated whole tumor cells are allogeneic.

43. The therapeutic vaccine of claim 37, wherein the whole tumor cells and the S180 cells or fragments thereof are inactivated by irradiation.

44. The therapeutic vaccine of claim 43, wherein the whole tumor cells and the S180 cells or fragments thereof are irradiated with radiation in an amount of about 25 to about 100 Gy.

45. The therapeutic vaccine of claim 37, wherein the whole tumor cells and the S180 cells or fragments thereof are inactivated with mitomycin.

46. The therapeutic vaccine of claim 45, wherein the amount of mitomycin is about 40 μg/ml.

47. The therapeutic vaccine of claim 37, wherein the vaccine comprises a mixture of inactivated whole tumor cells and inactivated S180 cells or fragments thereof.

48. The therapeutic vaccine of claim 37, wherein the vaccine comprises fusions of inactivated whole tumor cells and inactivated S180 cells or fragments thereof.
